(19) United States
(12) Patent Application Publication
(10) Pub. No.: US 2008/0286249 A1
(43) Pub. Date: Nov. 20, 2008

(54) USE OF MESENCHYMAL STEM CELLS FOR TREATING GENETIC DISEASES AND DISORDERS

(76) Inventors: Timothy R. Varney, Baltimore, MD (US); Charles Randal Mills, Finksburg, MD (US); Alla Danilkovitch, Columbia, MD (US)

Correspondence Address: MCANDREWS HELD & MALLOY, LTD, 500 WEST MADISON STREET, SUITE 3400, CHICAGO, IL, 60661, US

(21) Appl. No.: 12/042,487

(22) Filed: Mar. 5, 2008

Related U.S. Application Data
(63) Continuation-in-part of application No. 11/651,878, filed on Jan. 10, 2007.
(60) Provisional application No. 60/758,387, filed on Jan. 12, 2006.

(51) Int. Cl. A61K 35/12; A61P 37/02

(52) U.S. Cl. 424/93.21; 424/93.7

(57) ABSTRACT

A method of treating a genetic disease or disorder such as, for example, cystic fibrosis, Wilson's disease, amyotrophic lateral sclerosis, or polycystic kidney disease, in an animal comprising administering to said animal mesenchymal stem cells in an amount effective to treat the genetic disease or disorder in the animal.

### CROSS REFERENCE TO RELATED APPLICATIONS

This application is a continuation-in-part application of U.S. patent application Ser. No. 11/651,878, filed on Jan. 10, 2007, (currently pending), which claims priority to U.S. provisional application Ser. No. 60/758,387, filed Jan. 12, 2006, (now abandoned), the contents of each which are incorporated by reference in their entireties.

### BACKGROUND OF THE INVENTION

Mesenchymal stem cells (MSCs) are multipotent stem cells that can differentiate readily into lineages including osteoblasts, myocytes, chondrocytes, and adipocytes (Pittenger, et al., Science, vol. 284, pg. 143 (1999); Haynesworth, et al., Bone, vol. 13, pg. 69 (1992); Prockop, Science, vol. 276, pg. 71 (1997)). In vitro studies have demonstrated the capability of MSCs to differentiate into muscle (Wakitani, et al., Muscle Nerve, vol. 18, pg. 1417 (1995)), neuronal-like precursors (Woodbury, et al., J. Neurosci. Res., Vol, 69. pg. 908 (2002); Sanchez-Ramos, et al., Exp. Neurol., vol. 171, pg. 109 (2001)), cardiomyocytes (Toma, et al., Circulation, vol. 105, pg. 93 (2002); Fakuda, Artif. Organs, vol. 25, pg. 187 (2001)) and possibly other cell types. In addition, MSCs have been shown to provide effective feeder layers for expansion of hematopoietic stem cells (Eaves, et al., Ann. N.Y. Acad. Sci., vol. 938, pg. 63 (2001); Wagers, et al., Gene Therapy, vol. 9, pg. 606 (2002)).

Recent studies with a variety of animal models have shown that MSCs may be useful in the repair or regeneration of damaged bone, cartilage, meniscus or myocardial tissues (Dekok, et al., Clin. Oral Implants Res., vol. 14, pg. 481 (2003)); Wu, et al., Transplantation, vol. 75, pg. 679 (2003); Noel, et al., Curr. Opin. Investig. Drugs, vol. 3, pg. 1000 (2002); Ballas, et al., J. Cell. Biochem. Suppl., vol. 38, pg. 20 (2002); Mackenzie, et al., Blood Cells Mel. Dis., vol. 27 (2002)). Several investigators have used MSCs with encouraging results for transplantation in animal disease models including osteogenesis imperfecta (Pereira, et al., Proc. Nat. Acad. Sci., vol. 95, pg. 1142 (1998)), parkinsonism (Schwartz, et al., Hum. Gene Ther., vol. 10, pg. 2539 (1999)), spinal cord injury (Chopp, at al., Neuroreport, vol. 11, pg. 3001 (2000); Wu, at al., Neurosci. Res., vol. 72, pg. 393 (2003)) and cardiac disorders (Tomita, et al., Circulation, vol. 100, pg. 247 (1999); Shake, at al., Ann. Thorac. Surg., vol. 73, pg. 1919 (2002)).

Promising results also have been reported in clinical trials for osteogenesis imperfecta (Horwitz, et al., Blood, vol. 97, pg. 1227 (2001); Horowitz, at al. Proc. Nat. Acad. Sci., vol. 99, pg. 8932 (2002)) and enhanced engraftment of heterologous bone marrow transplants (Frassoni, et al., Int. Society for Cell Therapy, SA006 (abstract) (2002); Koc, et al., J. Clin. Oncol., vol. 18, pg. 307 (2000)).

### SUMMARY OF THE INVENTION

The present technology generally relates to mesenchymal stem cells. More particularly, the presently described technology relates to the use of mesenchymal stem cells for treating genetic diseases and disorders. Still more particularly, the present technology relates to the use of mesenchymal stem cells for treating genetic diseases or disorders that are characterized by inflammation of at least one tissue and/or at least one organ.

In at least one aspect, the present technology provides fort the use of MSCs for repopulating a host tissue with MSCs. Yet another aspect of the present technology provides for the use of MSCs for improving the function of dysfunctional tissue. Still more particularly, in yet another aspect of the present technology there is provided the use of mesenchymal stem cells for improving the function of dysfunctional tissue that is characterized by a genetic defect and/or inflammation or inflammatory mediators.

### BRIEF DESCRIPTION OF THE DRAWINGS

The following is a brief description of the drawings which are presented for the purposes of illustrating the present technology and not for the purposes of limiting the same.

FIG. 1 is a series of photomicrographs of colonies of mesenchymal stem cells derived from rat bone marrow following whole body irradiation and one of the following: control treatment, intraosseous delivery of exogenous bone marrow cells and mesenchymal stem cells, or intravenous delivery of exogenous bone marrow cells and mesenchymal stem cells. FIG. 1A shows human placental alkaline phosphatase (hPAP) stained cells. FIG. 1B shows cells stained with Evans blue.

### DETAILED DESCRIPTION OF THE INVENTION

It has been surprisingly discovered that mesenchymal stem cells, when administered systemically, such as by intravenous or intraosseous administration, migrate toward and engraft within inflamed tissue. Thus, in accordance with at least one aspect of the present technology, there is provided one or more methods of treating a genetic disease or disorder in an animal, more particularly, a method of treating a genetic disease or disorder that is characterized by at least one of an inflamed tissue or organ of the animal. In at least some embodiments, the method comprises the step of administering to the animal (including a human) mesenchymal stem cells in an amount effective to treat the genetic disease or disorder in the animal.

Although the scope of the present technology is not to be limited to any theoretical reasoning, infused mesenchymal stem cells (MSCs) home to, i.e., migrate toward, and engraft within inflamed tissue. Inflammatory involvement has been described for several genetic diseases including, but not limited to, polycystic kidney disease, cystic fibrosis, Wilson's Disease, Gaucher's Disease, and Huntington's Disease, for example. The presence of inflammation within the tissue or organs affected by these and other genetic disorders may facilitate homing of the MSCs to the inflamed tissues and/or organs, and facilitate engraftment of the MSCs.

Again, not wanting to be bound by any particular theory, it is believed that the administration of the MSCs may correct tissue and/or organ dysfunction caused by a genetic defect in that the MSCs carry a wild-type copy of the gene that is defective in the animal being treated. The administration of the MSCs to the patient (animal, including humans) results in the engraftment of cells that carry the wild-type gene to tissues and/or organs affected by the disease. The engrafted MSCs can differentiate according to the local environment. Upon differentiation, the MSCs can express the wild-type version of the protein that is defective or absent from the surrounding tissue. Engraftment and differentiation of the donor MSCs within the defective tissue and/or organ can correct the tissue and/or organ function.

As will be appreciated by one of skill in the art, MSCs may be genetically modified to contain a wild-type copy of the gene that is defective in the animal being treated. Alternatively, genetic transduction of the donor MSCs may not be required if, for example, the donor MSCs have an endogenous wild-type version of a gene that is defective in the animal being treated. Thus, it is believed that the correction of tissue and/or organ function results from the presence of such a wild-type gene(s).

Further, the use of MSCs as a vehicle for wild-type gene delivery can provide normal copies of all genes which, when mutated, lead to the development of the genetic disease to be treated. This is believed to be accomplished (1) whether the gene defect(s) has (have) been identified, (2) whether the contribution of the mutated form of the gene(s) to the development of the disease is known, or (3) whether the disease results from a single genetic mutation or a combination of genetic mutations. The expression of the normal form of the proteins which, when non-functional, contribute to the development of the disease, can improve or correct the function of tissues impaired by the disease.

In general, the genetic disease or disorder to be treated via the methods of the present technology is a genetic disease or disorder characterized by at least one inflamed tissue or organ, although other genetic diseases and disorders may be treated as well. Genetic diseases or disorders that may be treated in accordance with the presently described technology include, but are not limited to, cystic fibrosis, polycystic kidney disease, Wilson's disease, amyotrophic lateral sclerosis (or ALS or Lou Gehrig's Disease), Duchenne muscular dystrophy, Becker muscular dystrophy, Gaucher's disease, Parkinson's disease, Alzheimer's disease, Huntington's disease, Charcot-Marie-Tooth syndrome, Zellweger syndrome, autoimmune polyglandular syndrome, Marfan's syndrome, Werner syndrome, adrenoleukodystrophy (or ALD), Menkes syndrome, malignant infantile osteopetrosis, spinocerebellar ataxia, spinal muscular atrophy (or SMA), or glucose galactose malabsorption.

For example, cystic fibrosis (CF) is a genetic disorder characterized by impaired functionality of secretory cells in the lungs, pancreas and other organs. The secretion defect in these cells is caused by the lack of a functional copy of the Cystic Fibrosis Transmembrane Conductance Regulator (CFTR) gene. Mutations in the CFTR gene result in the appearance of an abnormally thick, sticky mucus lining in the lungs that clogs air passages and leads to life-threatening infections. Also, thick secretions in the pancreas prevent digestive enzymes from reaching the intestines, leading to poor weight gain, among other complications.

In some embodiments, MSC administration according to the present technology described herein may be employed to treat CF symptoms by providing wild type (normal) CFTR genes to tissues affected by the disease. It is believed that the localization of systemically delivered MSCs to the lungs is effected by both the path of circulatory flow and by the migration response of MSCs to inflamed tissues. CF patients typically suffer from frequent Pseudomonas aeruginosa infections of the lungs. Successive rounds of Pseudomonas infection and resolution are accompanied by inflammation and scarring. Inflammatory markers in the lungs of CF patients include TNF-α and MCP-1, chemokines that are known to promote MSC recruitment.

Thus, it is further believed that following the integration within affected tissues, the MSCs differentiate (mature) according to the local environment and begin producing functionally normal CFTR protein. The presence of cells containing an active form of the protein could improve or correct the secretory impairment observed in CF tissues. MSC delivery also may limit the progression of fibrosis and scar expansion in the lungs of CF patients (i.e., animals, including humans).

Wilson's disease is a genetic disorder of copper transport, resulting in copper accumulation and toxicity in the liver, brain, eyes and other sites. The liver of a person who has Wilson's disease does not release copper into the bile correctly. A defect in the ATP7B gene is responsible for the symptoms of Wilson's disease.

Copper accumulation in the liver results in tissue damage characterized by inflammation and fibrosis. The inflammatory response of Wilson's disease involves TNF-α, a chemokine known to promote the recruitment of MSCs to damaged tissue. Systemically delivered MSCs therefore are believed to migrate to regions of inflamed liver in Wilson's disease patients. Upon engraftment, the MSCs differentiate to form hepatocytes and initiate expression of the normal copy of the ATP7B gene and production of functional ATP7B protein. As a result, hepatocytes derived from exogenously delivered MSCs therefore may carry out normal copper transport, thereby reducing or ameliorating excess copper accumulation in the liver. Location-specific maturation of MSCs may reduce the buildup of copper in the brain and eyes as well. The reduction of copper accumulation in these tissues could resolve the symptoms of Wilson's disease in patients treated by MSC therapy.

Amyotrophic lateral sclerosis (ALS or Lou Gehrig's Disease) is a neurological disorder characterized by progressive degeneration of motor neuron cells in the spinal cord and brain, which results ultimately in paralysis and death. The SOD1 gene (or ALS1 gene) is associated with many cases of familial ALS (See, e.g., Nature, vol. 362:59-62). Again not wanting to be bound by any particular theory, it is believed that the enzyme coded for by SOD1 removes superoxide radicals by converting them into non-harmful substances. Defects in the action of SOD1 result in cell death due to excess levels of superoxide radicals. Thus, several different mutations in this enzyme all result in ALS, making the exact molecular cause of the disease difficult to ascertain. Other known genes that, when mutated, contribute to the onset of ALS include ALS2 (Nature Genetics, 29(2):166-73.), ALS3 (Am J Hum Genet, 2002 January; 70(1):251-6.) and ALS4 (Am J Hum Genet. June; 74(6).).

It is suspected that there are several currently unidentified genes that contribute to susceptibility to ALS. This is particularly the case in patients (e.g., human patients) with non-familial ALS. Thus, according to the usage and methodology of the present technology, it is believed that MSC treatment could provide normal copies of these genes to ALS patients because donor MSCs may be obtained from healthy donors and mutations that result in the development of ALS are rare.

As a result, according to the present technology, it is further believed that the use of MSCs as a vehicle for wild type gene delivery can provide normal copies of all genes which, when mutated, lead to the development of ALS. This is true (1) whether the gene defect(s) has been identified, (2) whether the contribution of the mutated form of the gene(s) to the development of ALS is known, and (3) whether the disease results from a single genetic mutation or a combination of genetic mutations. The expression of the normal form of the proteins which, when non-functional, contribute to the development of ALS could restore muscle function in ALS patients.

Muscular dystrophies are diseases involving progressive wasting of the voluntary muscles, eventually affecting the muscles controlling pulmonary function. Duchenne and Becker muscular dystrophies are both caused by mutations in the gene that encodes the protein dystrophin. In Duchenne's muscular dystrophy, the more severe disease, normal dystrophin protein is absent. In the milder Becker's muscular dystrophy, some normal dystrophin is made, but in insufficient amounts.

Dystrophin imparts structural integrity to muscle cells by connecting the internal cytoskeleton to the plasma membrane. Muscle cells lacking or having insufficient amounts of dystrophin also are relatively permeable. Extracellular components can enter these more permeable cells, this increasing the internal pressure until the muscle cell ruptures and dies. The subsequent inflammatory response can add to the damage. The inflammatory mediators in muscular dystrophy include TNF-α (Acta Neuropathol LBerl)., 2005 February; 109(2):217-25. Epub 2004 Nov. 16), a chemokine known to promote MSC migration to damaged tissue.

Thus, delivery of MSCs according to the present technology containing a normal dystrophin gene is believed to treat the symptoms of Duchenne's and Becker's muscular dystrophy in the following manner. MSC migration to degenerative muscle can result in MSC differentiation according to the local environment, in this case to form muscle cells. It is believed that MSCs that differentiate to form muscle will express normal dystrophin protein, because these cells carry the normal dystrophin gene. MSC-derived muscle cells could fuse with endogenous muscle cells, providing normal dystrophin protein to the multinucleated cell. The successful fusion of dystrophin-expressing MSCs with differentiating human myoblasts has been reported in an article entitled, “Human mesenchymal stem cells ectopically expressing full-length dystrophin can complement Duchenne muscular dystrophy myotubes by cell fusion.” (Goncalves, et al, Advance Access published online on Dec. 1, 2005 in Human Molecular Genetics.) The greater the degree of MSC engraftment within degenerative muscle, the closer the muscle tissue could resemble normal muscle structurally and functionally.

Gaucher's disease results from the inability to produce the enzyme glucocerebrosidase, a protein that normally breaks down a particular kind of fat called glucocerebroside. In Gaucher's disease, glucocerebroside accumulates in the liver, spleen, and bone marrow.

Gaucher's disease may be treated by the delivery of MSCs, for example, according to the methodology of the present technology, that harbor a normal copy of the gene that encodes glucocerebrosidase. Tissue damage caused by glucocerebroside accumulation produces an inflammatory response that causes the migration of MSCs to damaged regions. The inflammatory response in Gaucher's disease involves TNF-α, a cytokine known to recruit MSCs to areas of tissue damage (Eur Cytokine Netw., 1999 June; 10(2):205-10). Once engrafted within damaged tissue, MSCs could differentiate to replace missing cell types according to local environmental cues. MSC derived cells may have the ability to break down glucocerebroside normally, due to the ability to express active glucocerebrosidase by such cells. Thus, intravenously delivered glucocerebrosidase enzyme is effective in slowing the progression of, or even reversing the symptoms of Gaucher's disease (Biochem Biophys Res Commun., 2004 May 28; 318(2):381-90.). It is not known if wild type MSCs will produce glucocerebrosidase that will be available externally to the MSC-derived cell that produces the enzyme. If so, glucocerebrosidase expression by exogenously derived MSCs will reduce glucocerebroside levels in surrounding tissue. However, it is believed that the benefit of MSC therapy for Gaucher's disease in this manner would lie not only in the contribution of cells that have the ability to break down glucocerebroside, but also in the fact that these cells can provide glucocerebrosidase to neighboring cells as well, resulting in the reduction of glucocerebroside in native tissue.

Parkinson's disease (PD) is a motor system disorder that results from the loss of dopamine-producing brain cells. The primary symptoms of PD are tremor, stiffness of the limbs and trunk, bradykinesia, and impaired balance and coordination. A classic pathological feature of the disease is the presence of an inclusion body, called the Lewy body, in many regions of the brain.

It is believed, generally, that there is a genetic component to PD, and that a variety of distinct mutations may result in disease onset. One gene thought to be involved in at least some cases of Parkinson's is ASYN, which encodes the protein alpha-synuclein. The accumulation of alpha-synuclein in Lewy body plaques is a feature of both Parkinson's and Alzheimer's diseases.

However, it is not yet clear whether alpha-synuclein accumulation is a root cause of neural damage in Parkinson's or a result of neural cell death. If alpha-synuclein buildup is a primary cause of neural degeneration, then one possibility is that one or more additional proteins responsible for regulating the expression or accumulation of alpha synuclein damage has declined with age. One mechanism by which MSC therapy may treat PD therefore, is through providing a renewed source of one or more of such regulatory proteins.

Regardless of the genetic basis of the disease, it is believed that delivery of MSCs according to the present technology to PD patients could result in the replacement of dopamine-producing cells. Inflammation resulting from neuronal cell death should cause MSC migration directly to affected regions of the brain.

Alzheimer's disease results in a progressive loss in the ability to remember facts and events, and eventually to recognize friends and family. The pathology in the brains of Alzheimer's patients is characterized by the formation of lesions made of fragmented brain cells surrounded by amyloid-family proteins.

Delivery of MSCs, as according to the present technology, that contain normal copies of the presenilin-1 (PS1), presenilin-2 (PS2) and possibly other, as yet unidentified, genes is believed to treat the complications of Alzheimer's disease. The inflammation resulting from brain cell fragmentation that is characteristic of the disease attracts MSCs to migrate into the area. Then, MSCs can differentiate into neural cell types when located within damaged neural tissue. Further, the metalloproteinases expressed and secreted by MSCs reduces the characteristic lesions found in the brains of Alzheimer's patients by degrading amyloid proteins and other protein types within these plaques. Resolution of amyloid plaques could provide an opportunity for the differentiation of MSCs and endogenous stem cells to form neurons.

Huntington's disease (HD) is an inherited, degenerative neurological disease that leads to decreased control of movement, loss of intellectual faculties and emotional disturbance. A mutation in the HD gene, the gene that encodes the Huntingtin protein, eventually results in nerve degeneration in the basal ganglia and cerebral cortex of the brain.

How mutations in the HD gene result in Huntington's disease is currently not clear. The inflammation associated with neural degeneration, however, provides an environment that is conducive to MSC recruitment. MSC engraftment to these regions can lead to differentiation according to the local environment, including MSC maturation to form neurons that carry a normal form of the HD gene. One effect of MSC therapy, therefore, may be to replace neurons lost to neural degeneration. The delivery methodology according to the practice of the present technology is believed to accomplish such a result and/or outcome.

Contributing factors to the onset and/or progression of Huntington's disease may include an age-related decrease in regulatory proteins that control the production level of Huntingtin protein. Thus, the administration of MSCs is also believed to restore the availability of such regulatory constituents.

Charcot-Marie-Tooth syndrome (CMT) is characterized by a slow progressive degeneration of the muscles in the foot, lower leg, hand, and forearm and a mild loss of sensation in the limbs, fingers, and toes.

The genes that produce CMT when mutated are expressed in Schwann cells and neurons. Several different and distinct mutations, or combinations of mutations, can produce the symptoms of CMT. Different patterns of inheritance of CMT mutations are also known. One of the most common forms of CMT is Type 1A. The gene that is mutated in Type 1A CMT is thought to encode the protein PMP22, which is involved in coating peripheral nerves with myelin, a fatty sheath that is important in nerve conductance. Other types of CMT include Type 1B, autosomal-recessive, and X-linked.

Delivery of MSCs according to the present technology, for example, expressing a normal copy of the Type 1A CMT gene, Type 1B CMT gene and/or other genes may restore the myelin coating of peripheral nerves. A component of the inflammatory response in degenerative regions involves the production and secretion of MCP-1 (monocyte chemoattractant protein-1; J. Neurosci Res., 2005 Sep. 15; 81(6):857-64), a cytokine known to support the homing of MSCs to damaged tissue. The mechanism of restoring the structure and functionality of degenerative tissue will depend on the particular mutation involved in promoting the disease.

In Type I diabetes, the immune system attacks beta cells, the cells in the pancreas which produce insulin. The presence of certain genes, gene variants, and alleles may increase susceptibility to the disease. For example, susceptibility to the disease is increased in patients carrying certain alleles of the human leukocyte antigen (HLA) DQB1 and DRB1. Again, it is believed that the delivery of MSCs, according to the present technology, from a donor with normal copies of Type I diabetes susceptibility genes may restore the body's ability to manufacture and use insulin. Regardless of the genetic basis of the disease, delivery of MSCs to Type I diabetics may result in the replacement of dysfunctional insulin producing cells. The inflammatory markers present in the pancreas of type I diabetes patients include TNF-α, a chemokine known to attract MSCs. Therefore, systemically administered MSCs via the present technology may home to regions of inflamed pancreatic tissue in Type I diabetics. Upon engraftment the MSCs may differentiate into insulin-producing cells. Additionally, the MSC engraftment may protect insulin-producing beta cells from detection and destruction by the immune system. The restoration of beta cell number may resolve or reduce the severity of Type I diabetes.

Other genetic diseases that may be treated by administering MSCs according to the practice of the present technology are listed below.

Polycystic kidney disease: Delivery of a normal form of the PKD1 gene may inhibit cyst formation.

Zellweger syndrome: Delivery of a normal copy of the PXRI gene by the MSCs may correct peroxisome function, imparting normal cellular lipid metabolism and metabolic oxidation.

Autoimmune Polyglandular syndrome: The disease may be treated by delivery of MSCs expressing a normal copy of the ARE (autoimmune regulator) gene and/or regeneration of glandular tissue destroyed during disease progression.

Marfan's syndrome: Delivery of MSCs expressing a normal form of the FBN1 gene could result in the production of fibrillin protein. The presence of fibrillin may impart normal structural integrity to connective tissues.

Werner syndrome: Delivery of MSCs expressing normal form of the WRN gene could provide a source of cells for tissue turnover that do not age prematurely.

Adrenoleukodystrophy (ALD): Delivery of MSCs expressing a normal form of the ALD gene may result in correct neuron myelination in the brain and/or may lead to regeneration of damaged areas of the adrenal gland.

Menkes syndrome: Delivery of MSCs that express a normal copy of an as yet unidentified gene or genes on the X chromosome that have the capability of absorbing copper could resolve disease symptoms.

Malignant infantile osteopetrosis: MSCs could, for example, carry normal copies of genes that, when mutated, contribute to the onset of malignant infantile osteopetrosis. These genes include the chloride channel 7 gene (CLCN7), the osteopetrosis associated transmembrane protein (OSTM1) gene, and the T-cell immune regulatory (TCIRG1) gene. MSC delivery may correct the osteoblast/osteoclast ratio by providing MSCs that may act as osteoblast precursors and/or precursors to other cell types that control osteoclast differentiation.

Spinocerebellar ataxia: Delivery of MSCs that express a normal form of the SCA1 gene provides cells that can differentiate to form new neurons that produce the ataxin-1 protein (the product of the SCA1 gene) at appropriate levels to replace host neurons lost to neural degeneration. It is also possible that MSC engraftment may provide proteins that regulate the expression of the ataxin-1 protein.

Spinal muscular atrophy: Delivery of MSCs that express a normal copy of the SMA gene may provide cells that could differentiate to form new motor neurons to replace neurons that have died during disease progression.

Glucose galactose malabsorption: Delivery of MSCs expressing normal copies of the SGLT1 gene may correct glucose and galactose transport across the intestinal lining.

It will be appreciated by one of skill in the art that MSCs may be genetically modified to contain a wild-type copy of a gene. For example, the MSCs may be genetically modified to contain a gene, or a portion thereof, a combination, a derivative, or alternative thereof, such as, for example, the CFTR gene, the ATP7B gene, the SOD1 gene, the gene that encodes the protein dystrophin, the gene that encodes the protein glucocerebrosidase, the ASYN gene, the HD gene, the gene that encodes the protein PMP22, the PKD1 gene, the PXRI gene, the ARE gene, the FBN1 gene, the WRN gene, the ALD gene, the CLCN7 gene, the OSTM1 gene, the TCIRG1 gene, the SCA1 gene, the SMA gene, or the SGLT1 gene. As will be further appreciated by one of skill in the art, MSCs may be genetically modified to contain one or more exogenous genes. Such genetic modification may be effected by methods and techniques that are well-known in the art, including transfection and transformation.

It is to be understood, however, that the scope of the present technology described and claimed herein is not to be limited to the treatment of any particular genetic disease or disorder. Rather, it shall be appreciated by those skilled and familiar with the art that the present technology can be utilized in a variety of different manners in the delivery of MSCs.

Thus, in accordance with at least one aspect of the present technology, there is provided one or more methods for repopulating a host tissue (human or animal) with mesenchymal stem cells. The methods comprise the steps of reducing an endogenous mesenchymal stem cell population in the host and administering to the host isolated exogenous mesenchymal stem cells in an amount effective to repopulate the host tissue with mesenchymal stem cells. Thus, the repopulated tissue may comprise a mixture of exogenous MSCs and endogenous MSCs. Alternatively, the repopulated tissue may be substantially free of endogenous MSCs.

In accordance with another aspect of the presently described technology, there is provided one or more methods for improving the function of dysfunctional tissue in an animal (e.g., a human). The method comprises the step of administering to the animal mesenchymal stem cells in an amount effective to improve the function of dysfunctional tissue. The mesenchymal stem cells may be administered systemically, such as by intravenous or intraosseous delivery, or directly to the dysfunctional tissue. The dysfunctional tissue may be characterized by a genetic defect and/or inflammation and inflammatory mediators, including those that promote MSC migration to damaged tissue.

In accordance with a further aspect of the present technology, there is provided a pharmaceutical composition for improving the function of dysfunctional tissue in an animal (e.g., a human). The pharmaceutical composition comprises mesenchymal stem cells in an amount effective to improve the function of dysfunctional tissue. The dysfunctional tissue may be characterized by a genetic defect and/or inflammation and inflammatory mediators, including those that promote MSC migration to damaged tissue.

In at least one embodiment respective of this aspect, the animal to which the mesenchymal stem cells are administered is a mammal. The mammal may be a primate, including human and nonhuman primates.

Moreover, the mesenchymal stem cell (MSC) therapies, methods, compositions of the present technology are generally based, for example, on the following sequence: harvest of MSC-containing tissue, isolation and expansion of MSCs, and administration of the MSCs to the animal, with or without biochemical manipulation.

The mesenchymal stem cells that are administered according to the practice of the present technology may be a homogeneous composition or may be a mixed cell population enriched in MSCs. Homogeneous mesenchymal stem cell compositions may be obtained by culturing adherent marrow or periosteal cells, and the mesenchymal stem cells may be identified by specific cell surface markers which are identified with unique monoclonal antibodies. A method for obtaining a cell population enriched in mesenchymal stem cells is described, for example, in U.S. Pat. No. 5,486,359. Alternative sources for mesenchymal stem cells include, but are not limited to, blood, skin, cord blood, muscle, fat, bone, perichondrium, liver, kidney, lung and placenta.

The mesenchymal stem cells utilized in the performance of the present technology may be administered by a variety of procedures. For example, the mesenchymal stem cells may be administered systemically, such as by intravenous, intraarterial, intraperitoneal, or intraosseous administration. The MSCs also may be delivered by direct injection to tissues and organs affected by the disease. In one embodiment, the mesenchymal stem cells are administered intravenously. Thus, one of skill and having familiarity with the art will appreciate that the presently described technology can be administered in a variety of ways that are suitable for MSC delivery and for usage with MSC-based therapies. Additionally, one of skill and familiarity with the art will also appreciate that the present technology can be utilized in treatment modalities, systems, or regimens in which the MSCs are a component or an aspect or part of the modality, system, or regimen desired.

Additionally, the mesenchymal stem cells may be from a spectrum of sources, including allogeneic, autologous, and xenogeneic.

For example, in one embodiment of the present technology, prior to the administration of the donor mesenchymal stem cells, the host mesenchymal stem cell population is reduced, which increases donor MSC persistence. The host mesenchymal stem cell population may be reduced by any of a variety of means known to those skilled in the art, including, but not limited to, partial or full body irradiation, and/or chemoablative or nonablative procedures. This procedure has been shown previously to increase MSC migration to the bone marrow. Without wishing to be bound by any particular theory, it is believed that this procedure provides an open niche for donor MSC engraftment (tissue integration) according to the practice of the present technology.

In another non-limiting embodiment, the host mesenchymal stem cell population is reduced by any of a variety of means known to those skilled in the art, including, but not limited to those recited herein above. Host tissue then may be, repopulated by administration of the donor MSCs. Following administration of the donor MSCs, the host tissue MSC population may comprise greater than 50% donor or exogenously-derived cells. Alternatively, the host tissue MSC population may comprise greater than 80% donor or exogenously-derived cells. Alternatively, substantially all of the repopulated host tissue MSCs may be of donor origin or exogenously-derived.

Following administration of the allogeneic donor MSCs according to the present technology, the host tissue MSC population may be a mixture of host-derived MSCs and donor-derived MSCs. Alternatively, the host tissue MSC population may be substantially free of host-derived or endogenous MSCs.

In one non-limiting embodiment, the host is subjected to partial or full body irradiation prior to administration of the donor MSCs. The radiation may be administered as a single dose, or in multiple doses. For example in some embodiments, the radiation is administered in a total amount of from about 8 Grays (Gy) to about 12 Grays (Gy). In alternative embodiments, the radiation is administered in a total amount of from about 10 Gy to about 12 Gy. The amount of radiation to be administered and the number of doses administered are dependent upon a variety of factors, including the age, weight, and sex of the patient, and the general health of the patient at the time of administration.

In other non-limiting embodiments, when the host MSC population is reduced through partial or full body irradiation and/or chemoablative or nonablative procedures, hematopoietic stem cells are administered along with the MSCs in order to reconstruct the host's hematopoietic system. The hematopoietic stem cells may be derived from a variety of sources, including, but not limited to bone marrow, cord blood, or peripheral blood. The amount of hematopoietic stem cells to be administered is dependent on a variety of factors, including the age, weight, and sex of the patient, the radiation and/or chemoablative or nonablative treatment given to the patient, the general health of the patient, and the source of the hematopoietic stem cells.

In still further embodiments, the donor MSCs may be allogeneic to the host. The donor MSCs may be human leukocyte antigen (HLA) matched or mismatched to the host. The donor MSCs may be partially HLA-mismatched to the host. For example, the donor and host may be non-identical siblings. Without wishing to be bound by any particular theory, it is believed that allogeneic donor MSCs, including donor MSCs that are partially HLA-mismatched to the host, may increase the engraftment rate and persistence of donor MSCs under certain circumstances where donor hematopoietic stem cells are co-administered with MSCs to the patient. Co-administration of hematopoietic stem cells may be necessary to reconstitute the blood and immune system following procedures to reduce the patient's endogenous MSC population, as described above. The administration of MSCs and hematopoietic stem cells having an identical or substantially similar immunophenotype with respect to each other to a patient having a substantially dissimilar phenotype with respect to the donated MSCs and donated hematopoietic stem cells may promote engraftment and persistence of donor MSCs.

For example, the donor MSCs and donor hematopoietic stem cells both may be obtained from an HLA-matched sibling of the recipient. Alternatively, donor MSCs and donor hematopoietic stem cells are obtained from two donating individuals having a substantially similar immunophenotype with respect to each other, but a substantially dissimilar immunophenotype with respect to the patient. In either case, the reconstituted immune system derived from donated hematopoietic stem cells should not react with (reduce the numbers of) the donated MSCs, or should have a limited effect on reducing the numbers of donated MSCs. Under these conditions, the donated MSCs may have a survival advantage over host MSCs, thereby increasing the ratio of donor-derived MSCs to host MSCs in the treated patient.

In at least one embodiment of the present technology, the bone marrow cells, including hematopoietic stem cells, are autologous to the patient. In further embodiments, the autologous bone marrow cells are administered in an amount of from 1×10⁷ cells to about 1×10⁸ cells per kg of body weight.

In other embodiments, the bone marrow cells, including hematopoietic stem cells, are allogeneic to the patient. The donor bone marrow cells may be HLA-matched or HLA-mismatched to the host. The donor bone marrow cells may be partially HLA-mismatched to the host. For example, the donor and host may be non-identical siblings. In a further embodiment, the allogeneic bone marrow cells are administered in an amount of from about 1×10⁸ cells to about 3×10⁸ cells per kg of body weight.

Additionally, the mesenchymal stem cells utilized according to the present technology are administered in an amount effective to treat the genetic disease or disorder in an animal (e.g., a human). In at least one embodiment, the mesenchymal stem cells are administered in an amount of from about 0.5×10⁶ MSCs per kilogram (kg) of body weight to about 10×10⁶ MSCs per kg of body weight. In yet other embodiments, the mesenchymal stem cells are administered in an amount of about 8×10⁶ MSCs per kg of body weight. In further embodiments, the mesenchymal stem cells are administered in an amount of from about 1×10⁶ MSCs per kg of body weight to about 5×10⁶ MSCs per kg of body weight. In still further embodiments, the mesenchymal stem cells are administered in an amount of about 2×10⁶ MSCs per kg of body weight. Alternatively, the mesenchymal stem cells may also be administered at a flat dose of 200×10⁶ MSCs per infusion to an individual weighing about 35 kg or more, 50×10⁶ to an individual weighing less than about 35 kg, but weighing about 10 kg or more, and 20×10⁶ to an individual weighing less than about 10 kg, but weighing about 3 kg or more.

Moreover, the mesenchymal stem cells may be administered once, or the mesenchymal stem cells may be administered two or more times at periodic intervals of from about 3 days to about 7 days, or the mesenchymal stem cells may be administered chronically, i.e., during the entire lifetime of the animal (e.g., a human), at periodic intervals of from about 1 month to about 12 months. The amount of mesenchymal stem cells to be administered and the frequency of administration are dependent upon a variety of factors, including the age, weight, and sex of the patient (animal, including a human), the genetic disease or disorder to be treated, and the extent and severity thereof.

In accordance with another aspect of the present technology, there is provided a pharmaceutical composition for treating a genetic disease or disorder in an animal (e.g., a human). The pharmaceutical composition comprises mesenchymal stem cells in an amount effective to treat the genetic disease or disorder in the animal. The genetic disease or disorder may be characterized by at least one of an inflamed tissue or organ of the animal.

The mesenchymal stem cells may be administered with respect to this aspect of the present technology in conjunction with an acceptable pharmaceutical carrier. For example, the mesenchymal stem cells may be administered as a cell suspension in a pharmaceutically acceptable liquid medium for injection. In at least one embodiment, the pharmaceutically acceptable liquid medium is a saline solution. The saline solution may contain additional materials such as dimethylsufoxide (DMSO) and human serum albumin.

The presently described technology and its advantages will now be better understood by reference to the following examples. These examples are provided to describe specific embodiments of the present technology. By providing these specific examples, the applicant(s) do not intend in any manner to limit the scope and spirit of the present technology. It will be understood and appreciated by those skilled in the art that the full scope of the presently described technology includes the subject matter defined by the claims appending this specification, and any alternations, modifications, or equivalents of those claims.

### EXAMPLE 1

### Mesenchymal Stem Cells for Treatment of Cystic Fibrosis

Increased donor MSC persistence can be achieved by reducing the host MSC population through the use of full body irradiation and/or chemoablative or nonablative procedures before donor MSC delivery to the patient. This procedure provides an open niche for donor MSC engraftment (tissue integration) and has been shown previously to increase MSC migration to the bone marrow. In addition to MSC infusion, delivery of bone marrow cells or hematopoietic stem cells also will be required to reconstruct the patient's hematopoietic system, which may be destroyed by the methods used to reduce the number of host MSCs in the patient's bone marrow.

MSCs may be delivered by either intravenous infusion or injection directly to the bone marrow cavity (intraosseous injection). Although intravenous MSC delivery may be sufficient for successful MSC integration within the bone marrow of the recipient, intraosseous injection may enhance MSC engraftment persistence. Again, not wanting to be bound by any particular theory, it is believed that the rapid donor MSC engraftment should increase the likelihood that the exogenously-derived population will be well established before the expansion of any native MSCs that remain after host MSC reduction procedures.

A rat model of bone marrow transplant following irradiation is being used to test the hypothesis that either intravenous (IV) or intraosseous (IO). MSC delivery, concurrently with a bone marrow transplant, will result in engraftment following ablative procedures. The protocol also was designed to gain a preliminary comparative measure of the relative success of the two MSC delivery procedures.

On day 0, twelve male Lewis rats were irradiated with 2 fractions of 5.0 Grays (Gy). The radiation fractions were separated by 4 hours. On the following day, bone marrow cells (BMCs) were prepared from an additional 8-10 male Fisher rats. For injection, a total of 30×10⁶ BMCs and 1×10⁶ MSCs in a total volume of 150 ul were used. The MSCs used in the procedure carried the genetic marker human placental alkaline phosphatase (hPAP) for later detection. The experimental design for this study is shown in Table 1 below.

TABLE 1
Study Design. Allocation by experimental group.

| | Number of | | Total Body | |
|---|---|---|---|---|
| | Recipient | | Irradiation | |
| Group | Rats | Treatment | Day 1 | BMT Day 0 |
| 1 | 4 male | Control | 10 Gy* | none |
| | Lewis Rats | (no injection) | | |
| 2 | 4 male | Tibial Injection | 10 Gy* | 30 × 10⁶ |
| | Lewis Rats | (marrow + | | BM cells |
| | | hPAP cells) | | 1 × 10⁶ |
| | | | | hPAP MSCs |
| 3 | 4 male | IV infusion | 10 Gy* | 30 × 10⁶ |
| | Lewis Rats | (marrow + | | BM cells |
| | | hPAP cells) | | 1 × 10⁶ |
| | | | | hPAP MSCs |

*Radiation was divided into 2 fractions of 5.0 Gy. Radiation fractions were separated by 4 hours.

Animals in group 1 (control) received radiation only. Animals in group 2 were injected with MSCs and bone marrow cells directly into the head of the left tibia through the patellar ligament. Animals in group 3 were injected with MSCs and bone marrow cells intravenously.

The animals were weighed and observed daily for a period of 14 days, and any animal showing obvious signs of pain, such as head bobbing and/or writhing, was treated with buprenorphine. Buprenorphine was administered at a concentration of 0.5 mg/kg (of food) in 6 ml of soft daily food. This treatment started when the animals had lost 15% of their body weight and continued until scheduled euthanasia.

On day 14 all animals were sacrificed and bone marrow was collected from each tibia. The marrow samples were collected into tubes, sealed and packed in ice until they were plated out for assaying.

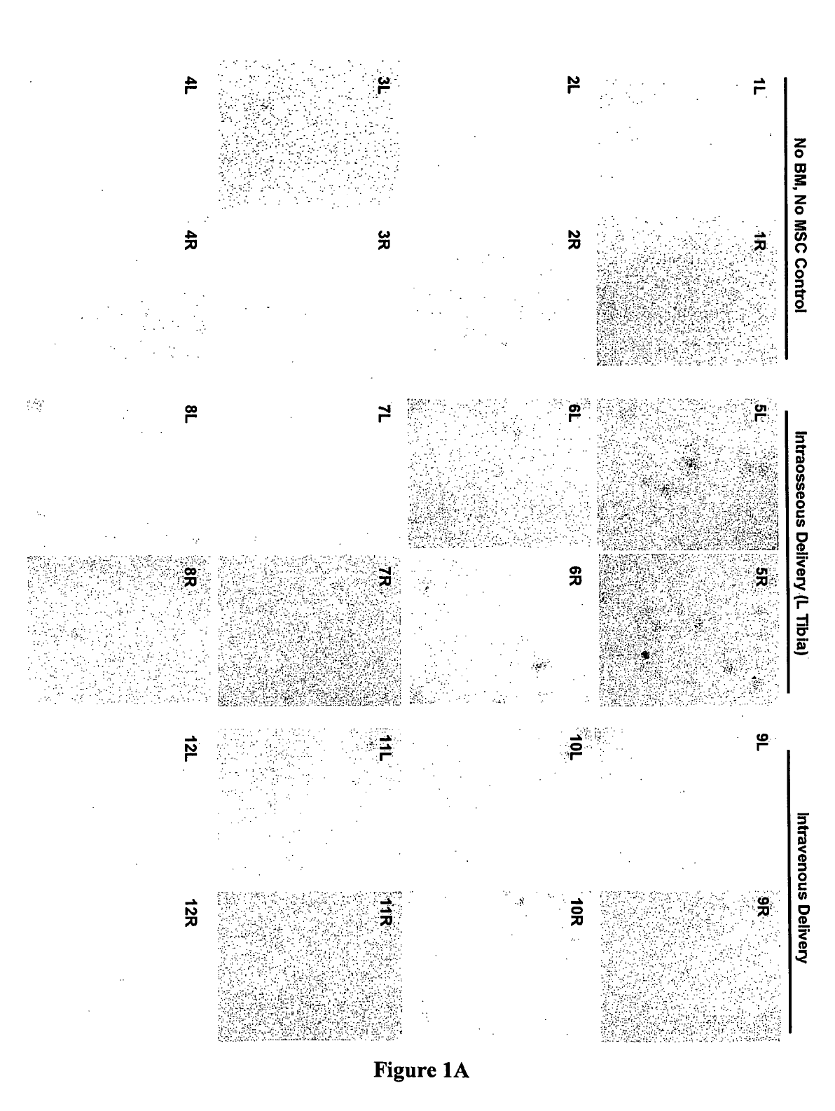

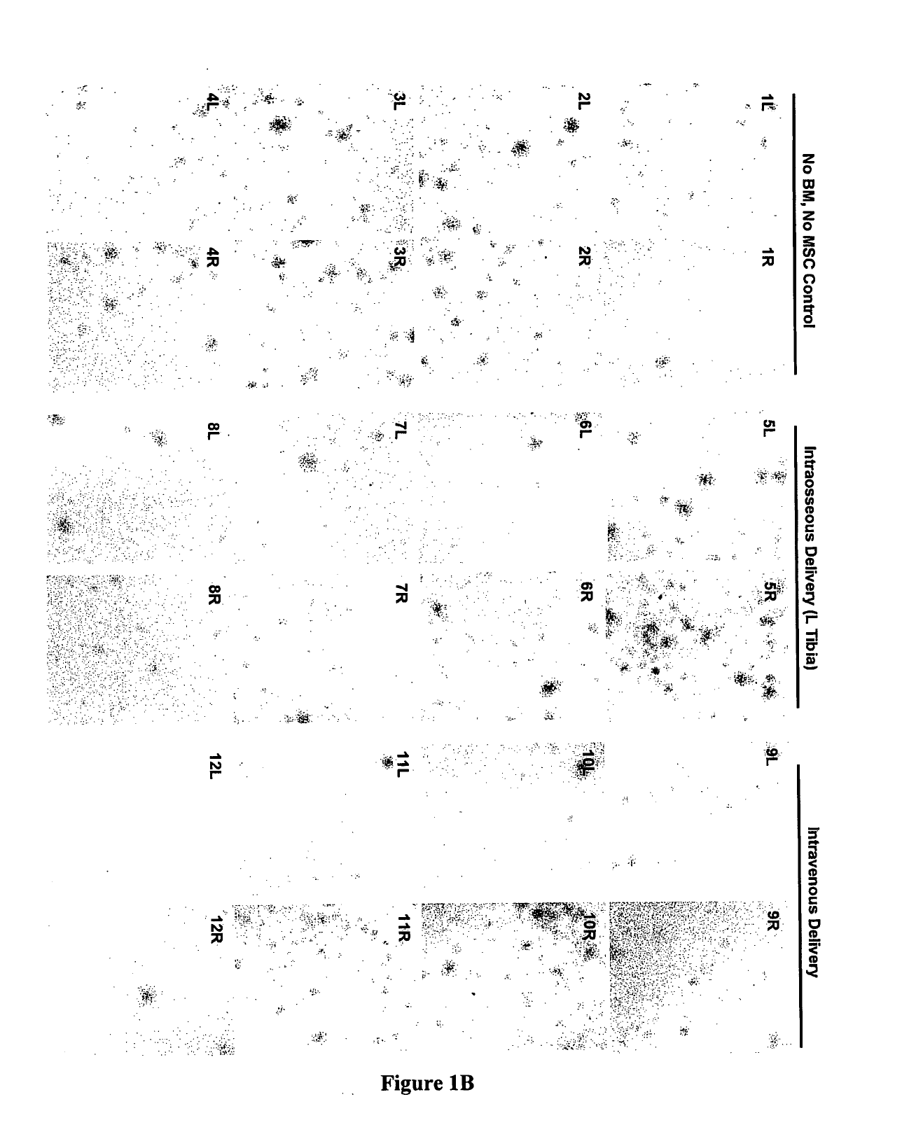

Bone marrow from each sample then was plated out for the colony forming unit assay. The cells were plated out at a low density, such that the formation of each colony was derived from the growth of a single MSC. The plated MSCs were left to grow for 12 days. Following this period of colony growth, plates were first stained for expression of the hPAP gene. Exogenously-derived MSC colonies on the plate were identified as pink-stained colonies (See FIG. 1A). Plates were then stained with Evans blue, which stains all colonies, whether derived from endogenous or exogenous MSCs, deep purple (See FIG. 1B). The percentage of MSCs derived from exogenous delivery could then be determined. The resulting data provides an initial assessment as to whether IV or IO delivery is more efficient in establishing the engraftment of donor-derived cells.

At 14 days post-transplant, approximately 100% of the colonies formed by mesenchymal stem cells derived from the bone marrow of animals in Groups 2 and 3 were comprised of exogenously-derived donor cells, as evidenced by hPAP staining (see FIG. 1A). Few, if any, colonies comprised recipient-derived cells (compare FIGS. 1A and 1B). In contrast, colonies comprised of recipient-derived cells were formed by mesenchymal stem cells derived from the bone marrow of animals in Group 1 (see FIG. 1B). Quite surprisingly, both IV and IO MSC delivery produce a high rate of initial engraftment. Additionally, IO and IV delivery of MSCs and BMCs (both HLA-identical with respect to each other, but partially HLA-mismatched with respect to the donor) appears to suppress or inhibit repopulation of the bone marrow with endogenous, or recipient-derived, MSCs. Thus, quite unexpectedly, it was found that up to the entire population of endogenous mesenchymal stem cells may be replaced by exogenously-derived mesenchymal stem cells.

Future studies could involve further investigation regarding the persistence and/or homing ability of transplanted MSCs in an animal model or the initiation of testing in human patients with genetic disease. Future studies in an animal model could include experimental subjects that are sacrificed at later time points post-transplantation. In this manner, the persistence of MSC engraftment is determined. The method of MSC delivery for these later experiments will be determined by pilot studies similar to that described above. Once the procedures for achieving persistent MSC engraftment have been developed in the rat model described above, a rat model of fibrotic lung injury is developed. Rats that have received an MSC transplant are given localized irradiation to the lungs. At various time points post irradiation, animals are sacrificed and the lungs are analyzed for the presence of MSCs by PCR or immunohistochemistry. The rat model described above in which experimental subjects with traceable MSCs are given localized radiation to the lungs is a surrogate for the fibrotic lung injury that occurs in cystic fibrosis. Significant migration of MSCs to the lungs following radiation injury in this rat model suggests that MSCs may participate in the healing of the fibrotic lung injury that is observed in cystic fibrosis patients.

The efficacy of MSC population replacement as a treatment for genetic disease can be evaluated in human patients in the following manner. A patient with (in this example) cystic fibrosis is given an intravenous infusion or an intraosseous injection of MSCs (2.5×10⁶ cells/ml) in PlasmaLyteA saline solution (Baxter) to which has been added DMSO at 3.75% vol./vol. and human serum albumin at 1.875% wt./vol. The infusion is continued until the patient receives a total of 2 million MSCs per kilogram of body weight. The treatment regimen is repeated at one month intervals. Lung function is assessed by spirometry. Treatment is continued until no further improvement in clinical symptoms is observed.

As discussed earlier herein, the underlying cause of fibrotic lung injury in patients who suffer from cystic fibrosis is a genetic defect. If MSCs are obtained from a genetically normal individual and transplanted to cystic fibrosis patients, then the migration of transplanted cells to the lungs in response to the inflammatory signals associated with fibrotic injury would result in an inhibition of the progression of the disease symptoms, or possibly even a reversal of clinical signs. The degree of improvement would be determined by the level of replacement of tissue lining the lungs. Thus, one of ordinary skill in the art can appreciate the significance of the present technology as a treatment modality, system or regimen for a cystic fibrosis, among other disease states and disorders.

### EXAMPLE 2

### Mesenchymal Stem Cells for Treatment of Wilson's Disease

The efficacy of MSC population replacement as a treatment for Wilson's disease can be evaluated in human patients in the following manner. The patient is given an intravenous infusion or an intraosseous injection of MSCs (2.5×10⁶ cells/ml) in PlasmaLyteA saline solution (Baxter) to which has been added DMSO at 3.75% vol./vol. and human serum albumin at 1.875% wt./vol. The infusion is continued until the patient receives 2 million MSCs per kilogram of body weight.

The treatment regimen is repeated at one month intervals, clinical symptoms are monitored by measuring serum ceruloplasmin, copper levels in the blood and urine, and imaging of the liver (i.e., abdominal X-ray or MRI). Treatment is continued until no further improvement in clinical symptoms is observed. Here again, the presently described technology is believed to provide a treatment modality, system, or regimen capable of providing a beneficial outcome in the prevention, treatment, or cure of Wilson's disease.

### EXAMPLE 3

### Mesenchymal Stem Cells for Treatment of Amyotrophic Lateral Sclerosis (ALS)

The efficacy of MSC population replacement as a treatment for Amyotrophic Lateral Sclerosis could be evaluated in human patients in the following manner. The patient is given an intravenous infusion or an intraosseous injection of MSCs (2.5×10⁶ cells/ml) in PlasmaLyteA saline solution (Baxter) to which has been added DMSO at 3.75% vol./vol. and human serum albumin at 1.875% wt./vol. The infusion is continued until the patient receives 2 million MSCs per kilogram of body weight.

The treatment regimen can be repeated at one month intervals. Clinical symptoms are monitored by neurological tests, electromyogram (EMG) to test muscle activity, and nerve conduction velocity (NCV) tests to evaluate nerve function. Treatment is continued until no further improvement in motor function is observed.

The present technology is now described in such full, clear, concise and exact terms as to enable any person skilled in the art to which it pertains, to practice the same. It is to be understood that the foregoing describes the preferred embodiments of the invention and that modifications may be made therein without departing from the spirit and scope of the present technology as set forth in the appended claims. Further, the disclosures of all patents, publications, including published patent applications, depository accession numbers, and database accession numbers are hereby incorporated by reference to the same extent as if each patent, publication, depository accession number, and database accession number were specifically and individually incorporated by reference.

What is claimed is:

1. A method for repopulating a host tissue with exogenous mesenchymal stem cells comprising the steps of:
reducing an endogenous mesenchymal stem cell population of a host tissue; and administering isolated exogenous mesenchymal stem cells in an amount effective to repopulate the host tissue with mesenchymal stem cells.

2. The method of claim 1, wherein the host tissue is bone marrow.

3. The method of claim 2, wherein the endogenous mesenchymal stem cell population is a population of bone marrow mesenchymal stem cells.

4. The method of claim 1, further comprising the step of administering exogenous bone marrow cells to the host.

5. The method of claim 4, wherein the bone marrow cells are allogeneic.

6. The method of claim 5, wherein the bone marrow cells are HLA-matched.

7. The method of claim 5, wherein the bone marrow cells are partially HLA-mismatched.

8. The method of claim 4, wherein the bone marrow cells are autologous.

9. The method of claim 1, wherein the repopulated tissue comprises exogenous mesenchymal stem cells and endogenous mesenchymal stem cells.

10. The method of claim 1, wherein the repopulated host tissue is substantially free of endogenous mesenchymal stem cells.

11. The method of claim 1, wherein the exogenous mesenchymal stem cells are allogeneic.

12. The method of claim 11, wherein the exogenous mesenchymal stem cells are HLA-matched or partially HLA-mismatched.

13. The method of claim 1, wherein the exogenous mesenchymal stem cells are autologous.

14. The method of claim 1, wherein the exogenous mesenchymal stem cells have been genetically modified.

15. The method of claim 14, wherein the exogenous mesenchymal stem cells have been genetically modified to contain a gene selected from the group consisting of the CFTR gene, the ATP7B gene, the SOD1 gene, the gene that encodes the protein dystrophin, the gene that encodes the protein glucocerebrosidase, the ASYN gene, the HD gene, the gene that encodes the protein PMP22, the PKD1 gene, the PXRI gene, the ARE gene, the FBN1 gene, the WRN gene, the ALD gene, the CLCN7 gene, the OSTM1 gene, the TCIRG1 gene, the SCA1 gene, the SMA gene, and the SGLT1 gene.

16. A method of improving the function of dysfunctional tissue comprising the step of administering isolated allogeneic mesenchymal stem cells in an amount effective to improve the function of the dysfunctional tissue.

17. The method of claim 16, wherein the dysfunctional tissue is characterized by a genetic defect.

18. The method of claim 16, wherein the dysfunctional tissue is characterized by inflammation.

19. The method of claim 16, wherein the allogeneic mesenchymal stem cells are administered by intravenous administration.

20. The method of claim 16, wherein the allogeneic mesenchymal stem cells are administered by intraosseous administration.

21. The method of claim 16, wherein the allogeneic mesenchymal stem cells are administered in an amount of from about 0.5×10⁶ cells per kilogram of body weight to about 10×10⁶ cells per kilogram of body weight.

22. The method of claim 16, wherein the allogeneic mesenchymal stem cells are administered in an amount of from about 1×10⁶ cells per kilogram of body weight to about 5×10⁶ cells per kilogram of body weight.

23. The method of claim 16, wherein the allogeneic mesenchymal stem cells are administered in an amount of about 2×10⁶ cells per kilogram of body weight.

24. A pharmaceutical composition for treating one or more genetic diseases or disorders in an animal comprising mesenchymal stem cells in an amount effective to treat the one or more genetic diseases or disorders in the animal.

25. The pharmaceutical composition of claim 24, wherein the genetic disease or disorder is characterized by at least one of an inflamed tissue or organ of the animal.

26. The pharmaceutical composition of claim 24, wherein the mesenchymal stem cells are allogeneic.

27. The pharmaceutical composition of claim 26, wherein the mesenchymal stem cells are HLA-matched or partially HLA-mismatched.

28. The pharmaceutical composition of claim 24, wherein the mesenchymal stem cells are autologous.

29. The pharmaceutical composition of claim 24, wherein the mesenchymal stem cells have been genetically modified.

30. The method of claim 29, wherein the exogenous mesenchymal stem cells have been genetically modified to contain a gene selected from the group consisting of the CFTR gene, the ATP7B gene, the SOD1 gene, the gene that encodes the protein dystrophin, the gene that encodes the protein glucocerebrosidase, the ASYN gene, the HD gene, the gene that encodes the protein PMP22, the PKD1 gene, the PXRI gene, the ARE gene, the FBN1 gene, the WRN gene, the ALD gene, the CLCN7 gene, the OSTM1 gene, the TCIRG1 gene, the SCA1 gene, the SMA gene, and the SGLT1 gene.

31. The pharmaceutical composition of claim 24, further comprising bone marrow cells.

32. A pharmaceutical composition for improving the function of dysfunctional tissue comprising isolated allogeneic mesenchymal stem cells in an amount effective to improve the function of the dysfunctional tissue.

33. The pharmaceutical composition of claim 32, wherein the dysfunctional tissue is characterized by a genetic defect.

34. The pharmaceutical composition of claim 33, wherein the dysfunctional tissue is characterized by the expression or production of inflammatory mediators.

35. The pharmaceutical composition of claim 34, wherein the mesenchymal stem cells are allogeneic.

36. The method of claim 35, wherein the exogenous mesenchymal stem cells are HLA-matched or partially HLA-mismatched.

37. The pharmaceutical composition of claim 34, wherein the mesenchymal stem cells are autologous.

38. The pharmaceutical composition of claim 34, wherein the mesenchymal stem cells have been genetically modified.

39. The method of claim 38, wherein the exogenous mesenchymal stem cells have been genetically modified to contain a gene selected from the group consisting of the CFTR gene, the ATP7B gene, the SOD1 gene, the gene that encodes the protein dystrophin, the gene that encodes the protein glucocerebrosidase, the ASYN gene, the HD gene, the gene that encodes the protein PMP22, the PKD1 gene, the PXRI gene, the ARE gene, the FBN1 gene, the WRN gene, the ALD gene, the CLCN7 gene, the OSTM1 gene, the TCIRG1 gene, the SCA1 gene, the SMA gene, and the SGLT1 gene.

40. The pharmaceutical composition of claim 34, further comprising bone marrow cells.
